(19) United States
(12) Patent Application Publication
(10) Pub. No.: US 2011/0224101 A1
(43) Pub. Date: Sep. 15, 2011

(54) Tumor associated proteome and peptidome analyses for multiclass cancer discrimination

(76) Inventors: Xuefeng Ling, Palo Alto, CA (US); James Schilling, San Mateo, CA (US); Liangbiao Chen, Beijing (CN); Jiagang Zhao, San Diego, CA (US)

(21) Appl. No.: 12/927,246

(22) Filed: Nov. 9, 2010

Related U.S. Application Data
(60) Provisional application No. 61/280,907, filed on Nov. 10, 2009.

(51) Int. Cl. C40B 40/10; G01N 33/574; H01J 49/26

(52) U.S. Cl. 506/18; 436/501; 435/7.4; 250/282

(57) ABSTRACT

Methods are provided for classification of cancer based on analysis of serologic biomarkers.

Although the term “cancer” is generally applied to certain hyperproliferative disorders, the differences between classes of cancer can be profound. While the term cancer generally refers to tumors that are made up of malignant cells capable of continuing cell divisions; of invading neighboring tissues; and of spreading, or metastasizing, to other areas of the body, classes of cancer can vary in cell or origin, drug sensitivity, metastatic potential, and other traits. An initial classifications of cancers might account for the differences in the cells from which the tumor is derived, where carcinomas derive from epidermal cells; leukemias and lymphomas derive from hematopoietic cells, sarcomas derive from bone or muscle, and so on. Yet even within these initial groupings there can be incredible diversity. It is therefore useful to have simple and effective tests that can distinguish one class of tumor from another.

Strategies for the treatment of cancer include reducing the initial incidence of cancer through prevention, and lowering mortality through early detection and treatment of tumors. Current efforts to combat cancer by a lack of effective clinical utilities for population screening, disease diagnosis, prognosis, monitoring of therapy, and prediction of therapeutic response. While advances in high throughput genomic and proteomic technologies have yielded potential DNA, RNA, and protein biomarker candidates under investigation for multiclass cancer classification, but the markers identified heretofore suffer from a number of drawbacks. To qualify as a practical serological diagnostic/prognostic utility, the biomarker should be stable and readily detectable in the circulation.

Serological markers may identify presence of primary tumors, or metastatic tumors. Other uses of serological markers include monitoring adherence to interventions and establishing trial outcomes. Serological markers have the potential for widespread applications. it is therefore of great interest to provide effective serological biomarkers and panels of biomarkers for cancer detection, and in particular markers that can distinguish different classes of cancer. The present invention addresses this need.

### SUMMARY OF THE INVENTION

Methods are provided for serological, multiclass discrimination of solid tumors. A patient sample is evaluated for the presence and relative levels of circulating protein or peptide cancer biomarkers selected from a panel of such proteins and peptides identified herein as indicative of a class of cancer. Different classes of cancer have a distinctive distribution profile of these biomarkers, and thus the distribution profile obtained from a patient sample is useful in rapidly and easily determining the class of cancer present in the individual from which the sample was taken. As the class of cancer is significant in determining initial assessment, e.g. biopsy, staging, etc., and in therapeutic approaches, the multiclass discrimination of the invention is useful in guiding patient therapy.

In some embodiments of the invention, the classes of cancer that are discriminated by the methods of the invention include, without limitation, colon cancer, kidney cancer, liver cancer and brain cancer.

In one embodiment of the invention, the panel of biomarkers comprises at least 10, usually at least 12 proteins, the presence of which may be assessed by antibody based assays, e.g. ELISA, RIA, etc., by quantitative mass spectrometry based approach for practical clinical utilities in serological diagnosis and prognosis, and the like. A protein biomarker panel of interest includes, without limitation, albumin (ALB), serotransferrin (TF), aplipoprotein A1 (APO A1), Vimentin (VIM), immunoglobulin heavy constant gamma 1 (IGHG1), glial fibrillary acidic protein (GFAP), alpha 1 antitrypsin (A1AT), hemoglobin beta (HBB), orosomucoid 1 (AGP1, alias ORM1), pyruvate kinase type M2 (PKM2, alias M2-PK), keratin 8 (KRT8), and keratin 19 (KRT19). Additional markers and clinical indicia may also be included in the analysis.

In another embodiment of the invention, the panel of biomarkers comprises at least 50, usually at least 53 peptides, including, without limitation, those peptides identified in Table 1 herein. The peptide panel includes tryptic peptides: a disintegrin and metalloproteinase domain 8 (ADAMS), orosomucoid 2 (AGP2, alias ORM2), immunoglobulin kappa constant (IGKC), MKI67 (FHA domain) interacting nucleolar phosphoprotein (MK1671P), tyrosine 3-monooxygenase/tryptophan 5-monooxygenase activation protein zeta polypeptide (YWHAZ), and non-tryptic peptides: ankyrin repeat and SOCS box-containing 13 (ASB13), Cyclin-J, glycoprotein Ib (platelet) alpha polypeptide (GP1BA), immunoglobulin superfamily, member 8 (IGSF8), RUN and EWE domain containing 4 (RUFY4), transient receptor potential cation channel subfamily M member 6 (TRPM6), and zinc finger and SCAN domain containing 4 (ZSCAN4), the presence of which may be assessed by antibody based assays, e.g. ELISA, RIA, etc., by quantitative mass spectrometry based approach for practical clinical utilities in serological diagnosis and prognosis, and the like. Additional markers and clinical indicia may also be included in the analysis, and analysis of the peptide panel may be combined with the protein panel described herein.

In another embodiment, prognostic algorithms are provided, which combine the results of multiple cancer biomarker level determinations and/or other clinical and laboratory parameters, and which utilizes multiclass discrimination of cancer types to provide a patient with a determination of cancer class from a serologic sample. In certain embodiments cancer biomarker distribution patterns are analyzed in combination with clinical, imaging, laboratory and genetic parameters to assess an individual patient's disease state and thereby determine if they would benefit from initiation of therapy. The use of such panels can provide a level of discrimination not found with individual cancer biomarkers.

In one use of such an algorithm, a reference dataset is obtained, which comprises, as a minimum, cancer biomarker binding profiles for representative cancer classes. Such information is provided herein, for example in FIG. 2, FIG. 4 and Table 1. Such a database may include positive controls representative of disease subtypes, and may also include negative controls, e.g. measurements of cancer biomarkers in normal human serum. The dataset optionally includes a profile for clinical indices; additional protein distribution patterns; metabolic measures, genetic information, and the like. The disease dataset is then analyzed to determine statistically significant matches between datasets, usually between reference datasets and test datasets and control datasets. Comparisons may be made between two or more datasets.

In other embodiments of the invention a device or kit is provided for the analysis of patient samples. Such devices or kits will include reagents that specifically identify the sets of cancer biomarkers identified herein. Devices of interest include arrays, where the reagents are spatially separated on a substrate such as a slide, gel, multi-well plate, etc. Alternatively the reagents may be provided as a kit comprising reagents in a suspension or suspendable form, e.g. reagents bound to beads suitable for flow cytometry, and the like. Reagents of interest include reagents specific for cancer biomarker markers.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Study design for the tumor associated biomarker discovery. NSC: nearest shrunken centroid feature selection algorithm; LDA: linear discriminative analysis.

FIG. 2. Mass spectrometric spectrum counting analysis qualitatively evaluates the relative abundance of the tumor associated proteins in colon (CC), kidney (KC), liver (LC) and brain (BC) tumors.

FIG. 3. Statistical evaluation of the effectiveness of the multiclass tumor classifications. Left panel: With the 868 feature peptide biomarker panel, the predicted discriminant probabilities of the CC, KC, LC, BC classes for each sample were calculated from the linear discriminant analysis (LDA). The maximum estimated probability for each of the wrongly classified samples is marked with a red arrow. Right panel: Goodness of separation for each tested nearest shrunken centroid (NSC) classifiers. The overall predictions and the robustness of the separation using various classifiers were analyzed by the spread of the distribution of the goodness of the separation, using a “box” (25%˜75%) and “whiskers” to break down data by percentile. For each panel, whisker plots for CC, KC, LC, and BC were generated.

FIG. 4. Analysis of the 53 peptide biomarker panel. Left panel: With the 53 feature peptide biomarker panel, the predicted discriminant probabilities of the CC, KC, LC, BC classes for each sample were calculated from the linear discriminant analysis (LDA). The maximum estimated probability for each of the wrongly classified samples is marked with a red arrow. Right panel: unsupervised two dimensional clustering of all CC, KC, LC and BC samples and the corresponding 53 peptide biomarkers. Heatmap reveals relative abundance of these 53 peptide biomarkers in the CC, KC, LC and BC tumor categories.

FIG. 5. Comparative analysis and heatmap plot of the relative abundance of the 53 peptide biomarkers in either the kidney or liver tumor tissue and their corresponding adjacent normal tissue counterpart isolated from the same patient.

FIG. 6. Alteration of proteolytic and anti-proteolytic networks has been proposed as the mechanism by which tumor associated protein and peptide expression patterns are generated.

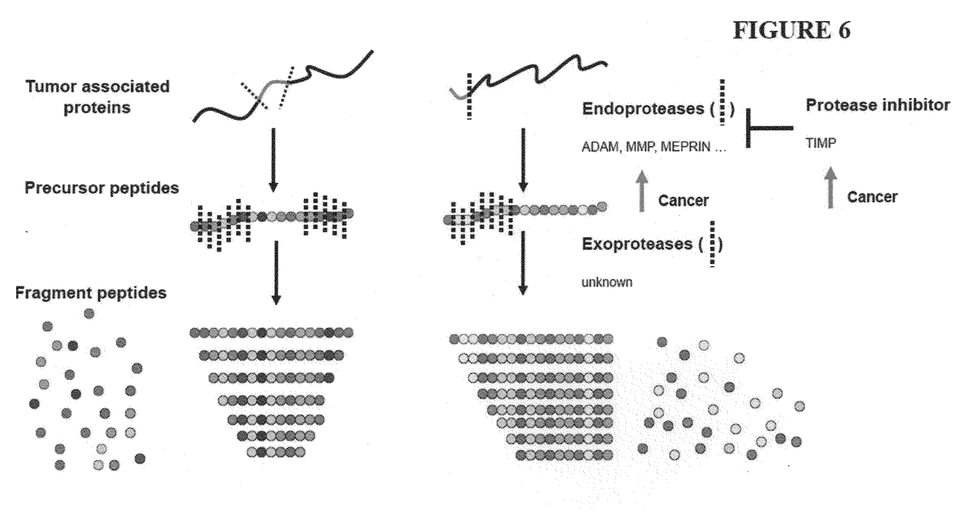

FIG. 7. Interrogation of the NCBI GEO database revealed that HBB is up-regulated in renal clear cell carcinoma.

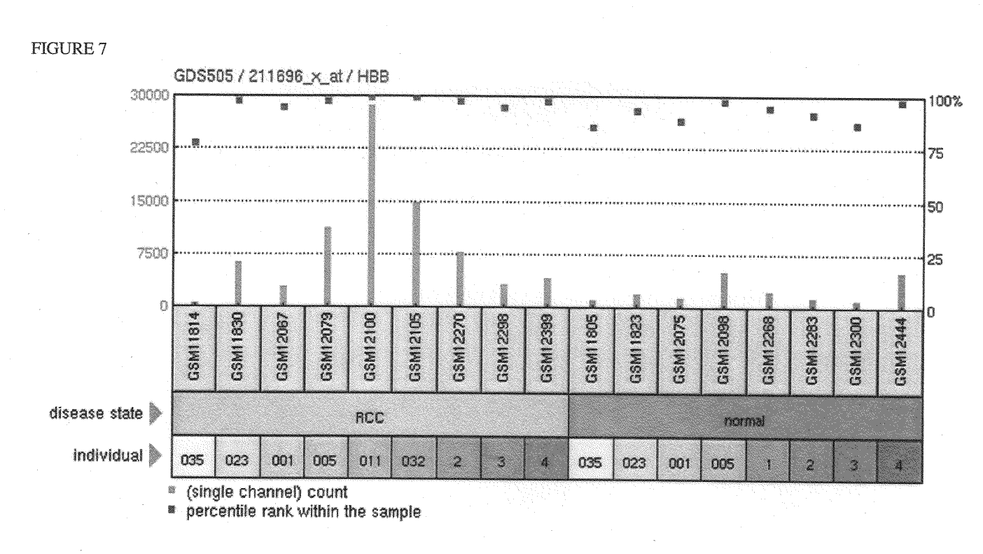

FIG. 8. Statistical evaluation of the effectiveness of the multiclass tumor classifications.

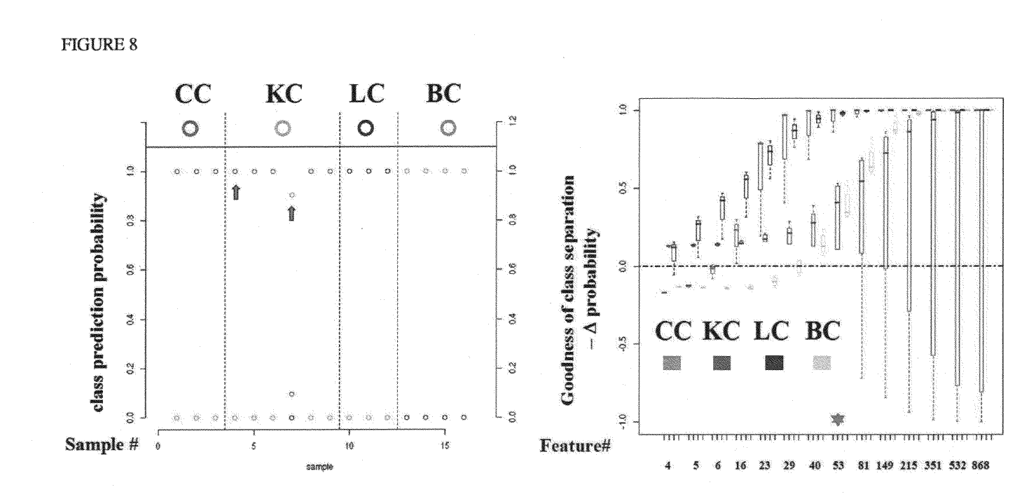

### DETAILED DESCRIPTION OF THE EMBODIMENTS

Methods are provided for serological, multiclass discrimination of solid tumors. A patient sample is evaluated for the relative levels of a panel of circulating proteins or peptides. Different classes of cancer have a distinctive distribution profile of these biomarkers, and thus the distribution profile obtained from a patient sample is useful in rapidly and easily determining the class of cancer present in the individual from which the sample was taken. As the class of cancer is significant in determining initial assessment, e.g. biopsy, staging, etc., and in therapeutic approaches, the multiclass discrimination of the invention is useful in guiding patient therapy.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges; and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to methods of classification of cancers, as well as reagents and kits for use in practicing the subject methods. The methods may also determine an appropriate level of treatment for a particular cancer.

Methods are also provided for optimizing therapy, by first classification, and based on that information, selecting the appropriate therapy, dose, treatment modality, etc. which optimizes the differential between delivery of an anti-proliferative treatment to the undesirable target cells, while minimizing undesirable toxicity. The treatment is optimized by selection for a treatment that minimizes undesirable toxicity, while providing for effective anti-proliferative activity.

The invention finds use in the prevention, treatment, detection or research into solid cancers. “Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and the like.

A “host cell”, as used herein, refers to a cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The term “normal” as used in the context of “normal cell,” is meant to refer to a cell of an untransformed phenotype or exhibiting a morphology of a non-transformed cell of the tissue type being examined.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

As used throughout, “modulation” is meant to refer to an increase or a decrease in the indicated phenomenon (e.g., modulation of a biological activity refers to an increase in a biological activity or a decrease in a biological activity).

Multiclass discrimination. As used herein, multiclass discrimination refers to the ability to determine which type of cancer is present in an individual based on analysis of serological markers as described herein. Cancer of particular interest for multiclass discrimination include solid tumors, which comprise, without limitation, colorectal cancer, hepatocellular cancer, gliomas, and renal cell carcinomas.

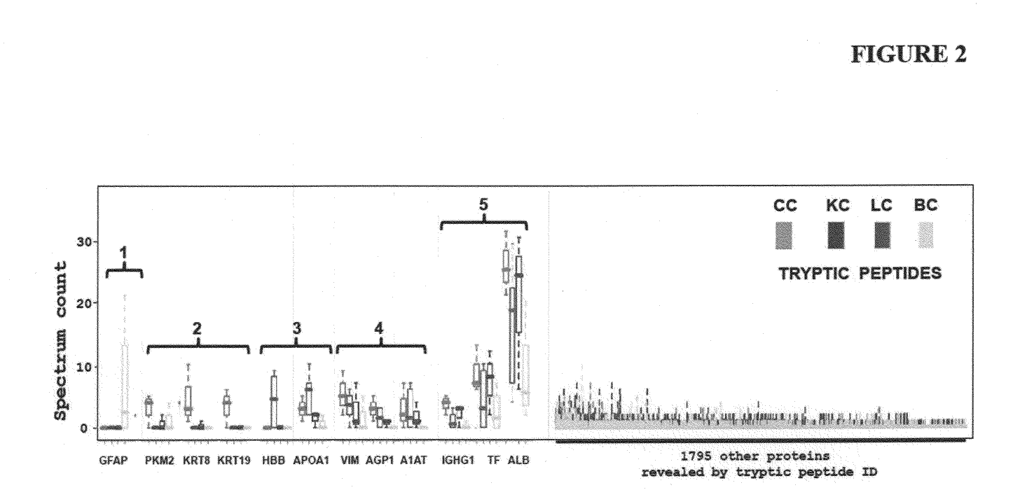

Protein Cancer Biomarker Panel. As used herein a protein biomarker panel comprises without limitation, albumin (ALB), serotransferrin (TF), aplipoprotein A1 (APO A1), Vimentin (VIM), immunoglobulin heavy constant gamma 1 (IGHG1), glial fibrillary acidic protein (GFAP), alpha 1 antitrypsin (A1AT), hemoglobin beta (HBB), orosomucoid 1 (AGP1, alias ORM1), pyruvate kinase type M2 (PKM2, alias M2-PK), keratin 8 (KRT8), and keratin 19 (KRT19). The relative distribution of these markers in serological samples from different cancer classes is shown in FIG. 2. The genetic sequences of these proteins are known in the art and accessible in public databases, such as Genbank.

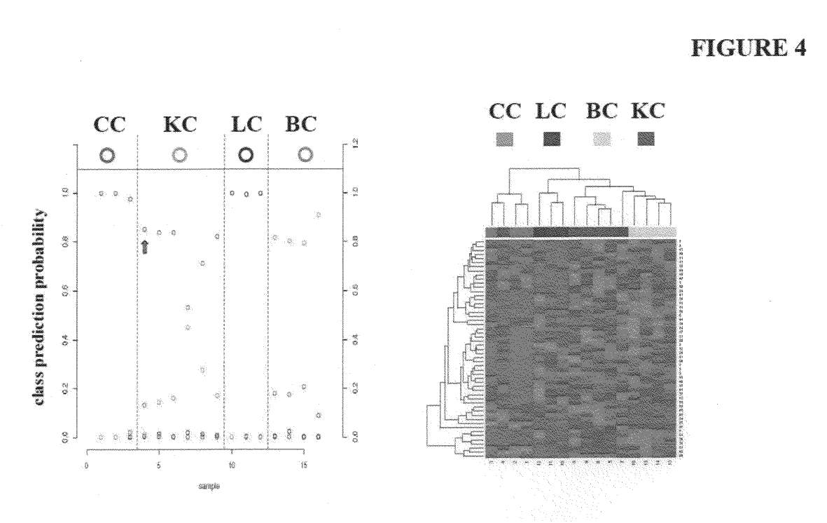

Peptide Cancer Biomarker Panel. As used herein a peptide biomarker panel comprises without limitation, those peptides identified in Table 1 herein. The peptide panel includes tryptic peptides: a disintegrin and metalloproteinase domain 8 (ADAMS), orosomucoid 2 (AGP2, alias ORM2), immunoglobulin kappa constant (IGKC), MKI67 (FHA domain) interacting nucleolar phosphoprotein (MKI671P), tyrosine 3-monooxygenase/tryptophan 5-monooxygenase activation protein zeta polypeptide (YWHAZ), and non-tryptic peptides: ankyrin repeat and SOCS box-containing 13 (ASB13), Cyclin-J, glycoprotein Ib (platelet) alpha polypeptide (GP1BA), immunoglobulin superfamily, member 8 (IGSF8), RUN and FYVE domain containing 4 (RUFY4), transient receptor potential cation channel subfamily M member 6 (TRPM6), and zinc finger and SCAN domain containing 4 (ZSCAN4). The relative distribution of these markers in serological samples from different cancer classes is shown in FIG. 4.

Certain of the peptides have been identified by amino acid sequence, as shown in Table 1. Other peptides are identified by mass spectrometry characteristics, also as set forth in Table 1.

Colorectal cancer (CRC) is extremely common, accounting for an estimated 153,000 cases and 52,000 deaths in the US annually. In Western countries, the colon and rectum account for more new cases of cancer per year than any anatomic site except the lung. Incidence begins to rise at age 40 and peaks at age 60 to 75. Overall, 70% of cases occur in the rectum and sigmoid, and 95% are adenocarcinomas. Prognosis depends greatly on stage.

CRC most often occurs as transformation within adenomatous polyps. Serrated adenomas are particularly aggressive in their malignant transformation. About 80% of cases are sporadic, and 20% have an inheritable component. Predisposing factors include chronic ulcerative colitis and granulomatous colitis; the risk of cancer increases with the duration of these disorders. CRC spreads by direct extension through the bowel wall, hematogenous metastasis, regional lymph node metastasis, perineural spread, and intraluminal metastasis.

Current methods for early diagnosis depend on routine examination, particularly fecal occult blood (FOB) testing. Patients with positive FOB tests require colonoscopy, as do those with lesions seen on sigmoidoscopy or imaging study. Elevated serum carcinoembryonic antigen (CEA) levels are present in 70% of patients with CRC, but this test is not specific and therefore is not recommended for screening. CA 199 and CA 125 are other tumor markers.

Surgery for cure can be attempted in the 70% of patients presenting without metastatic disease. Attempt to cure consists of wide resection of the tumor and its regional lymphatic drainage with reanastomosis of bowel segments.

Hepatocellular Carcinoma. Hepatocellular carcinoma (hepatoma, or liver cancer) usually occurs in patients with cirrhosis and is common in areas where infection with hepatitis B and C viruses is prevalent. Symptoms and signs are usually nonspecific. Diagnosis is based on α-fetoprotein (AFP) levels, imaging tests, and sometimes liver biopsy. Screening with periodic AFP measurement and ultrasonography is sometimes recommended for high-risk patients. Prognosis is poor when cancer is advanced, but for small tumors that are confined to the liver, ablative therapies are palliative and surgical resection or liver transplantation is sometimes curative.

For single tumors <5 cm or ≦3 tumors ≦3 cm that are limited to the liver), liver transplantation results in a good prognosis. Alternatively, surgical resection may be done; however, the cancer usually recurs. Ablative treatments (eg, hepatic arterial chemoembolization, intratumoral ethanol injection, cryoablation, radiofrequency ablation) provide palliation and slow tumor growth; they are used when patients are awaiting liver transplantation.

Renal Cell Carcinoma. (Adenocarcinoma of the Kidney, kidney cancer). Renal cell carcinoma (RCC), an adenocarcinoma, accounts for 90 to 95% of primary malignant renal tumors. In the US, about 51,000 cases of RCC and 8,000 deaths occur each year. Symptoms usually do not appear until late, when the tumor may already be large and metastatic. Gross or microscopic hematuria is the most common manifestation, followed by flank pain, FUO, and a palpable mass.

Most often, a renal mass is detected incidentally during abdominal imaging done for other reasons. Otherwise, diagnosis is suggested by clinical findings and confirmed by abdominal CT before and after injection of a radiocontrast agent or by MRI. A renal mass that is enhanced by radiocontrast strongly suggests RCC. CT and MRI also provide information about local extension and nodal and venous involvement.

Five-year survival rates range from 95% for the AJCC stage grouping I (T1 N0 M0) to 20% for stage grouping IV (T4 with any N or M; or N2 with any T or M; or M1). Prognosis is poor for patients with metastatic or recurrent RCC because treatments are usually ineffective for cure, although they may be useful for palliation.

Gliomas. (Brain cancer) Gliomas are primary tumors that originate in brain parenchyma. Gliomas include astrocytomas, oligodendrogliomas, medulloblastomas, and ependymomas. Many gliomas infiltrate brain tissue diffusely and irregularly. Astrocytomas are the most common gliomas.

Low-grade or anaplastic astrocytomas tend to develop in younger patients and can evolve into glioblastomas (secondary glioblastomas). Glioblastomas contain chromosomally heterogeneous cells. They can develop de novo (primary glioblastomas), usually iii middle-aged or elderly people. Primary and secondary glioblastomas have distinct genetic characteristics, which can change as the tumors evolve. Some astrocytomas contain oligodendroglioma cells; patients with these tumors (called oligoastrocytomas) have a better prognosis than those with pure astrocytomas.

Treatment involves surgery, radiation therapy, and chemotherapy to reduce tumor mass. After surgery, patients receive a full tumor dose of radiation therapy (60 Gy over 6 wk); ideally, conformal radiation therapy, which targets the tumor and spares normal brain tissue, is used.

### Methods of Classification

Compositions and methods are provided for classification of cancer patients according to the class of cancer, e.g. colon, kidney, liver or brain cancer, based on the distribution of serum biomarkers, as defined above. The distribution and quantitation of polypeptide biomarker may assessed by antibody based assays, e.g. ELISA, RIA, etc., by quantitative mass spectrometry based approach for practical clinical utilities in serological diagnosis and prognosis, and the like. Additional markers and clinical indicia may also be included in the analysis, and analysis of the peptide panel may be combined with the protein panel described herein.

As used herein, the term “polypeptide distribution pattern”, which may include distribution of proteins or peptides, refers to the quantitation and relative concentration of cancer biomarker levels in a patient sample. Once the polypeptide distribution pattern is determined, the information is used to classify the patient according to type of cancer, which classification is used in selecting the most appropriate therapy for an individual. Thus, the multiclass discrimination can provide information to guide clinical decision making, both in terms of institution of and escalation of therapy as well as in the selection of the therapeutic agent to which the patient is most likely to exhibit a robust response.

Mammalian species that provide samples for analysis include canines; felines; equines; bovines; ovines; etc. and primates, particularly humans. Animal models, particularly small mammals, e.g. murine, lagomorpha, etc. may be used for experimental investigations. Animal models of interest include those for models of autoimmunity, graft rejection, and the like.

Various techniques and reagents find use in the methods of the present invention. In one embodiment of the invention, blood samples, or samples derived from blood, e.g. plasma, serum, etc. are assayed for the presence of specific biomarkers. Typically a blood sample is drawn, and a derivative product, such as plasma or serum, is tested. Such biomarkers may be detected through specific binding members. Various formats find use for such assays. Many such methods are known to one of skill in the art, including ELISA, protein arrays, eTag system, bead based systems, tag or other array based systems etc. Examples of such methods are set forth in the art, including, inter alia, chip-based capillary electrophoresis: Colyer et al. (1997) J Chromatogr A. 781(1-2):271-6; mass spectroscopy: Petricoin et al. (2002) Lancet 359: 572-77; eTag systems: Chan-Hui et al. (2004) Clinical Immunology 111:162-174; microparticle-enhanced nephelometric immunoassay: Montagne et al. (1992) Eur J Clin Chem Clin Biochem. 30(4):217-22; antigen arrays: Robinson et al. (2002) Nature Medicine, 8:295-301; the Luminex XMAP bead array system (www.luminexcorp.com); and the like, each of which are herein incorporated by reference. Detection and quantitation may utilize one or a panel of specific binding members, e.g. specific for at least about 10, usually at least about 12 proteins, or for peptide analysis, usually at least about 50, at least about 53 or more peptides.

Cancer biomarker distribution patterns typically utilize a detection method coupled with analysis of the results to determine if there is a statistically significant match with a pre-determined pattern of interest.

The invention provides for methods of classifying tumors, and thus grouping or “stratifying” patients, according to the cancer biomarker distribution. As shown in the Examples, tumors classified as having a particular class of cancer may be analyzed and treated accordingly, and thus the methods provide for guidance of therapeutic options.

The polypeptide distribution pattern may be generated from a biological sample using any convenient protocol, for example as described below. The readout may be a mean, average, median or the variance or other statistically or mathematically-derived value associated with the measurement. The biomarker readout information may be further refined by direct comparison with the corresponding reference or control pattern. A pattern may be evaluated on a number of points: to determine if there is a statistically significant change at any point in the data matrix; whether the change is an increase or decrease in the biomarker concentration or distribution; whether the change is specific for one or more physiological states, and the like. The absolute values obtained for each biomarker under identical conditions will display a variability that is inherent in live biological systems.

Following obtainment of the polypeptide distribution pattern from the sample being assayed, the polypeptide distribution pattern is compared with a reference or control profile to make a classification regarding the cancer of the patient from which the sample was obtained. Typically a comparison is made with a sample or set of samples from an unaffected, normal source, and from a sample of known cancer class.

For multiplex analysis of cancer biomarkers, arrays containing one or more anti-cancer biomarker antibodies can be generated. Such an array is constructed comprising antibodies against cancer biomarkers, and may include antibodies binding cancer biomarkers listed in Table 1. Various immunoassays designed to quantitate cancer biomarkers may be used in screening. Measuring the concentration of the target protein in a sample or fraction thereof may be accomplished by a variety of specific assays. For example, a conventional sandwich type assay may be used in an array, ELISA, RIA, bead array, etc. format. A sandwich assay may first attach specific biomarkers to, an insoluble surface or support. The particular manner of binding is not crucial so long as it is compatible with the reagents and overall methods of the invention.

Arrays provide a convenient high throughput technique that can assay a large number of polypeptides in a sample. In one aspect of the invention, an array is constructed comprising antibodies specific for the panel of proteins or peptides described herein, preferably comprising antibodies specific for at least 10, or at least 12 distinct proteins, or at least 50, at least 53 distinct peptides as set forth in Table 1. This technology is used as a tool to quantitate the presence of cancer biomarkers in a sample. Arrays can be created by spotting antibodies onto a substrate (e.g., glass, nitrocellulose, etc.) in a two-dimensional matrix or array having bound probes. The antibodies can be bound to the substrate by either covalent bonds or by non-specific interactions, such as hydrophobic interactions. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Common physical substrates for making arrays include glass or silicon slides, magnetic particles or other micro beads, functionalized with aldehyde or other chemical groups to help immobilize proteins. The substrate can also be coated with PLL, nitrocellulose, PVDF membranes or modified with specific chemical reagents to adsorb capture agents. The desirable properties of an ideal surface include: chemical stability before, during, and after the coupling procedure, suitability for a wide range of capture agents (e.g., hydrophilic and hydrophobic, low MW and high MW), minimal non-specific binding, low or no intrinsic background in detection, presentation of the capture agents in a fully-functional orientation, production of spots with predictable and regular morphology (shape, signal uniformity).

In another embodiment, arrays of antibodies are attached to fluorescently addressable beads or other addressable tags. Antibodies are incubated with the addressable beads or tags to conjugate them via covalent bonds, avidin-biotin binding, electrostatic forces or other binding mechanisms. Such an approach may be performed using the Beadlyte Human 22-Plex Detection System (Upstate Biotechnology, Lake Placid, N.Y., USA) in conjunction with the Luminex 100 LabMAP System (Luminex, Austin, Tex., USA) for multiplex cancer biomarker analysis.

Both direct labeling and sandwich format approaches may find use. In the direct labeling procedure, the antibody array is interrogated with serum samples that had been derivatized with a fluorescent label, e.g. Cy3, Cy5 dye, etc. In the sandwich assay procedure, unlabeled serum is first incubated with the array to allow target biomarkers to be captured by immobilized capture antigens. Next, the captured biomarkers are detected by the application of a labeled detection reagent. The sandwich assay provides extra specificity and sensitivity needed to detect small concentrations of antibodies, without compromising the binding affinities of the antibodies through a direct labeling procedure.

Fluorescence intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

Other methodologies also find use. Methods such as surface plasmon resonance (SPR) are useful for label-free detection of antibody binding events, and can be applied in an array format to profile the distribution of cancer biomarkers. SPR senses refractive index change of molecules bound to a metal surface, and thereby enables detection of cancer biomarker binding using resonance and without need for fluorescent tags, enzymatic reactions, secondary antibodies, or washing methods that are frequently used in an immunoassay. In some embodiments, a solution based methodology utilizes capillary electrophoresis (CE) and microfluidic CE platforms for detecting and quantitating protein-protein interactions, including antibody reactions with serum cancer biomarker proteins and peptides. This technique can be performed easily by any laboratory with access to a standard CE DNA sequencing apparatus. With this methodology, a fluorescent marker (eTag reporter) is targeted to the analyte with one antibody, and a second sandwich antibody of different epitope specificity that is chemically coupled to a “molecular scissors” induces release of the fluorescent probe when both antibodies are in close apposition on the specific analyte. Quantitation then is focused on the liberated eTag, that is quantified with a standard DNA capillary sequencing device. The eTag Assay System can be used to measure the abundance of multiple proteins simultaneously. A critical feature of the assay is that the affinity agents (antibodies) are not immobilized on surfaces, as is required with array technologies. Solution-based binding eliminates surface-induced denaturation and non-specific binding, and improves sensitivity and reaction kinetics.

### Kits and Devices

The detection reagents can be provided as part of a kit. Thus, the invention further provides kits for detecting the presence and distribution of cancer biomarkers in a biological sample. Procedures using these kits can be performed by clinical laboratories, experimental laboratories, medical practitioners, or private individuals. The kits of the invention for detecting biomarkers may comprise antibodies useful for generating a cancer biomarker distribution pattern, which may be provided in solution or bound to a substrate. The kit may optionally provide additional components that are useful in the procedure, including, but not limited to, buffers, developing reagents, labels, reacting surfaces, means for detection, control samples, standards, instructions, and interpretive information. Devices of interest include arrays as described above. Alternatively the reagents may be provided as a kit comprising reagents in a suspension or suspendable form, e.g. reagents bound to beads suitable for flow cytometry, and the like. Reagents of interest include reagents specific for cancer biomarker markers. Such reagents may include cancer biomarker-specific antibodies or fragments thereof; and the like.

The kits may further include a software package for statistical analysis of one or more phenotypes, and may include a reference database for calculating the probability of classification. The kit may include reagents employed in the various methods, such as devices for withdrawing and handling blood samples, antibodies, ELISA reagents; tubes, spin columns, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that may be present is a website address which may be used via the internet to access the information at a removed site. Any convenient means may be present in the kits.

### Classification Algorithms

An algorithm that combines the results of multiple cancer biomarker level and distribution determinations, and controls for confounding variables and evaluating potential interactions is used for prognostic and diagnostic purposes. In such an algorithm, a cancer biomarker distribution pattern is obtained as a dataset. The dataset comprises quantitative data for the presence in serum of at least 10 cancer biomarkers, usually at least 12 proteins including, without limitation, albumin (ALB), serotransferrin (TF), aplipoprotein A1 (APO A1), Vimentin (VIM), immunoglobulin heavy constant gamma 1(IGHG1), glial fibrillary acidic protein (GFAP), alpha 1 antitrypsin (A1AT), hemoglobin beta (HBB), orosomucoid 1 (AGP1, alias ORM1), pyruvate kinase type M2 (PKM2, alias M2-PK), keratin 8 (KRT8), and keratin 19 (KRT19) and/or 53 peptides as set forth in Table 1. The dataset optionally quantitative data for the presence in a clinical sample of other markers, including the presence of additional cancer biomarkers, clinical indices, and the like.

In order to identify the class of cancer associated with a particular test sample, a statistical test will provide a confidence level for the distribution and concentration of biomarkers between the test and control profiles to be considered significant, where the control profile may be for one or multiple classes of cancer. The raw data may be initially analyzed by measuring the values for each marker, usually in duplicate, triplicate, quadruplicate or in 5-10 replicate features per marker.

A test dataset is considered to be match a control class of cancer distribution profile if at least 3, usually at least 5, at least 10, at least 15 or more of the parameter values of the profile match the limits that correspond to a predefined level of significance.

The data may be subjected to non-supervised hierarchical clustering to reveal relationships among profiles. For example, hierarchical clustering may be performed, where the Pearson correlation is employed as the clustering metric. One approach is to consider a patient cancer disease dataset as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Other methods of analysis that may be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

To provide significance ordering, the false discovery rate (FDR) may be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). This analysis algorithm is currently available as a software “plug-in” for Microsoft Excel know as Significance Analysis of Microarrays (SAM). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value may be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is. The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

Also provided are databases of cancer biomarker distribution patterns for classes of cancer. Such databases will typically comprise distribution patterns of the different cancer classes, where such profiles are as described above.

The analysis and database storage may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. Such data may be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The distribution patterns and databases thereof may be provided in a variety of media to facilitate their use. “Media” refers to a manufacture that contains the distribution pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

The following examples are offered by way of illustration and not by way of limitation. It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

### EXPERIMENTAL

### Example 1

Tumor associated proteins and peptides (TAP) are derived from tumor cells through apoptosis/necrosis, cell secretion or tumor-specific degradation of extracellular matrix proteins. In this study, primary tumor samples from colon cancer, kidney cancer, liver cancer, glioblastoma were analyzed by liquid chromatography coupled with mass spectrometry to identify these TAP biomarkers. Spectrum counting and peptidomic analyses found a 12-protein and a 53-peptide biomarker panels, capable of multiclass cancer detection and classification. If further validated prospectively in circulation, these TAP biomarkers have the potential to be developed into practical serological diagnostic and prognostic utilities.

The rationalebehind the present invention is that TAPs secreted by cancer cells or shed from the cancer microenvironment can enter the circulation, and that these proteins serological abundance can be assessed in combination with a biostatistics model for cancer prediction. Analysis of these TAPs, trapped in the source tumor tissues just prior to their release in circulation, can result in the discovery of even lower abundance, tissue specific, circulating biomarkers. The proteomic profiling analyses on these tumor associated proteins/peptides were taken directly from the primary tumor tissues.

Following surgical resection, tumor tissues were extensively rinsed, cut into small pieces, and incubated in the defined medium overnight at 4° C. The tissue conditioned media were expected to be enriched with proteins and peptides derived from tumor apoptosis/necrosis, secretion or tumor-specific degradation products of extracellular matrix proteins. Using spectrum counting method, mass spectrometric based proteomic profiling analysis found a biomarker panel of 12 proteins, having differential abundance between different tumor types. The naturally occurring serum or plasma peptidome has been the focus of recent attempts to find novel peptide biomarkers enabling highly accurate cancer class predictions [6; 9; 10]. These naturally occurring peptide biomarkers fall into tight clusters and that most are generated by exopeptidase activities that confer cancer type specific differences superimposed on the proteolytic events of the ex vivo coagulation and complement degradation pathways. We hypothesize that cancer microenvironment can generate and shed naturally occurring but tumor specific peptides via secreting tumor specific proteases or protease inhibitors therefore degrading or inhibiting the degradation of the surrounding abundant proteins and/or extracellular matrix. Thus comprehensive peptidomic analysis has been performed to overly and compare all the mass spectrometric spectra from various tumor samples for differential tumor associated peptide signals.

From 4 assayed tumor types—colon cancer, kidney cancer, liver cancer, and glioblastoma, a panel of 53 biomarkers was identified, including both tryptic peptides and non-tryptic peptides, which are capable of discriminating between these cancer types. These findings have important implications with regard to the validation and utilization of these tumor associated protein and peptide biomarkers as serological diagnostic and prognostic utilities to manage cancer.

### Results

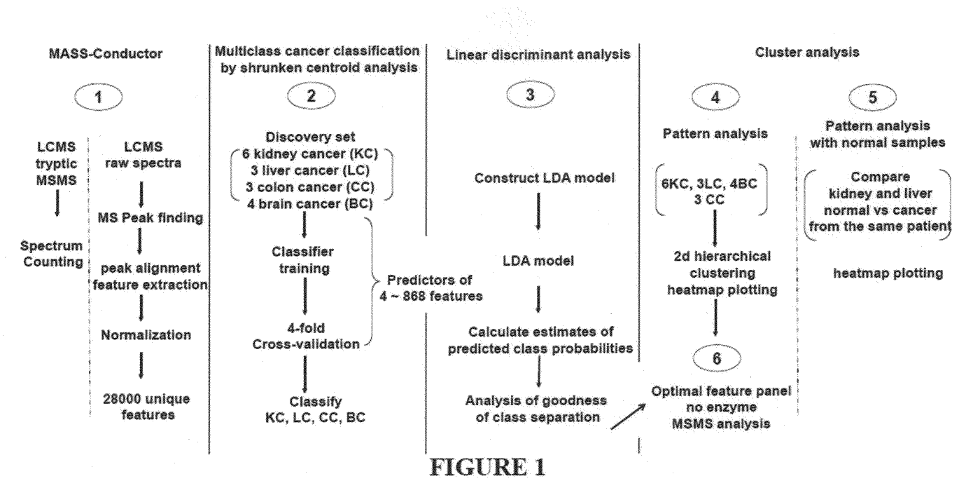

In this study (outlined in FIG. 1), we collected a total of 16 archived tissue samples from consenting cancer patients. The 16-case sample group contained primary tumor samples from 3 colon cancer, 6 kidney cancer, 3 liver cancer, 4 glioblastoma cancer patients, and tumor adjacent tissue counterparts from one kidney and one liver cancer patients. Following surgical resection, the tumor and the control tissues were cut into small pieces and extensively rinsed with PBS. To extract TAPs trapped in the tumor tissue, tissue specimens were kept at 4° C. overnight in defined medium such that tissue associated and/or extracellular matrix associated proteins and peptides can be released for subsequent extraction. After sample preparation and tryptic digestion, the peptides from the conditioned media were fractionated through C¹⁸ reverse phase HPLC and later analyzed by an LTQ FT mass spectrometry (MS).

Based upon tryptic peptide mass finger printing, a total of 1807 proteins were identified from control and tumor samples after searching the MS/MS spectra against the Swiss-Prot human database. Protein identifications from individual search engine results of all tumor samples were combined using probabilistic protein identification algorithms implemented in the Scaffold software. Spectrum counts were analyzed from the number of MS/MS spectra identified corresponding to each protein normalized to account for protein length or expected number of tryptic peptides. For any given protein, the relative abundance between samples was estimated by the comparative analysis of the normalized spectrum counts. The box-whisker graphs in FIG. 2 illustrate the spread of the distribution of the spectrum counts for each identified protein, using a “box” (25%˜75%) and “whiskers” to break down data by percentile.

The results show that 12 identified proteins, including albumin (ALB), serotransferrin (TF), aplipoprotein A1 (APO A1), Vimentin (VIM), immunoglobulin heavy constant gamma 1(IGHG1), glial fibrillary acidic protein (GFAP), alpha 1 antitrypsin (A1AT), hemoglobin beta (HBB), orosomucoid 1 (AGP1, alias ORM1), pyruvate kinase type M2 (PKM2, alias M2-PK), keratin 8 (KRT8), and keratin 19 (KRT19), had differential abundance in colon, kidney, liver and brain tumors. The remaining 1795 proteins' abundance was largely undifferentiated in those compared tumors.

Careful examination of the 12 differentially expressed proteins found that they can be divided into 5 groups of expression patterns (FIG. 2). In group 1, GFAP was the only protein of higher abundance in brain tumor than that in colon, kidney and liver tumors. In contrast, the remaining 11 proteins were all of lower abundance in brain than those in the other three tumors. PKM2, KRT8, KRT19 (group 2) were found to be highly expressed in colon cancer and were largely unexpressed in kidney, liver and brain tumor types. HBB and APO A1 (group 3) had more abundance in kidney tumor than in colon, liver and brain tumor types. VIM, AGP1 and A1AT (group 4) were found to be more expressed in colon tumor, then kidney tumor, then liver tumor and least in brain tumor. TF and ALB (group 5) were more abundant in colon and liver tumor than those in kidney and brain tumors.

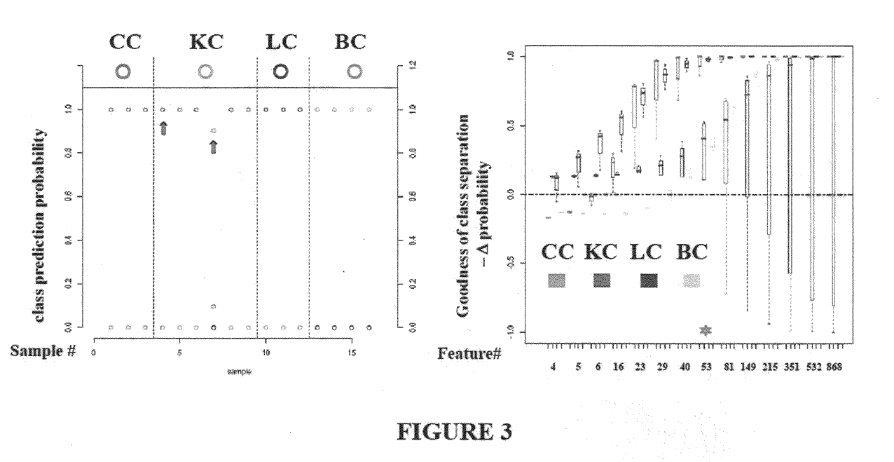

In addition to the identity-based spectrum counting analysis, a comprehensive analysis was performed comparing all MS scans to discover differential tryptic and non-tryptic peptide biomarkers. The non-tryptic peptides are likely to be the result of the tumor specific degradation of extracellular matrix proteins by proteases and exopeptidase released from cancer cells. The peptidomic analysis treats each of the LTQ FTMS peaks as distinct peak features. When applied to each HPLC fraction MS spectrum from different sample tumor categories, “MASS-Conductor” software extracts peaks from raw spectra, enables common peak alignment, generates “consensus” representative peaks across all spectra via two dimensional hierarchical clustering of both mass/charge and the HPLC fractions, and normalizes peak signal measurements. A total of 28000 unique peak features with distinct m/z and HPLC fraction have resolved. The samples were utilized as a training set (CC, n=3; KC, n=6; LC, n=3; BC, n=4) for predictor discovery. Predictor discovery by a nearest shrunken centroid (NSC) algorithm was performed with all the features in the data set. Four fold internal cross validation analysis led to the discovery of a set of 868 features with the minimum cross validation classification error. Discriminant class probabilities similar to Gaussian linear discriminant analysis (LDA) were calculated for each sample as previously described. FIG. 3 (left panel) displays these probabilities made by the 868 panel, where samples have robust separation between the highest and next highest probability, demonstrating that the sample is unambiguously classified into tumor categories. With the maximum estimated probability marked with red arrows, two of the KC samples are wrongly predicted.

In order to find a predictive biomarker panel of optimal feature number, balancing the need for small size panel, the accuracy of the overall prediction, robustness of the class separation, and good statistics of sensitivity and specificity, classifiers were built with a number of subsets of the 868 features. In this study, the goodness of separation is defined by computing the difference of the discriminative scores (estimated probability): if predicted correctly, Δ probability is the difference of the highest and next highest probability; if predicted incorrectly, Δ probability is the difference of the true class's probability and the highest probability, which will be negative. The box-whisker graphs in FIG. 3 (right panel) illustrate the spread of the distribution of the goodness of the separation, using a “box” (25%˜75%) and “whiskers” to break down data by percentile, demonstrating the overall predictions and the robustness of the separation using various classifiers. For each panel, whisker plots for CC, KC, LC, and BC were generated. The analysis of the goodness of separation revealed 53 to be the smallest panel size, where the “box” values of goodness of separation of all tumor categories remain positive. Therefore, this 53 feature panel was chosen as the peptide biomarker panel with predictive utility for follow up analysis. The peptide panel included both tryptic peptides: a disintegrin and metalloproteinase domain 8 (ADAM8), orosomucoid 2 (AGP2; alias ORM2), immunoglobulin kappa constant (IGKC), MKI67 (FHA domain) interacting nucleolar phosphoprotein (MKI671P), tyrosine 3-monooxygenase/tryptophan 5-monooxygenase activation protein zeta polypeptide (YWHAZ), and non-tryptic peptides: ankyrin repeat and SOCS box-containing 13 (ASB13), Cyclin-J, glycoprotein Ib (platelet) alpha polypeptide (GP1BA), immunoglobulin superfamily, member 8 (IGSF8), RUN and FYVE domain containing 4 (RUFY4), transient receptor potential cation channel subfamily M member 6 (TRPM6), and zinc finger and SCAN domain containing 4 (ZSCAN4), capable of discriminating between the colon, kidney, liver, and brain tumors.

FIG. 4 (left panel) displays these probabilities where tumor samples have robust separation between the highest and next highest probability, demonstrating that the most of the samples were correctly classified into CC, KC, LC and BC tumor categories using the 53 peptide panel. With the maximum estimated probability marked with a red arrow, only one of the KC samples was wrongly classified. Consistent with these findings, unsupervised clustering (FIG. 4, right panel) based upon the 53 peptide biomarker panel was able to largely cluster, according to their diagnosis, only one of the brain tumor was clustered within the colon cancer samples.

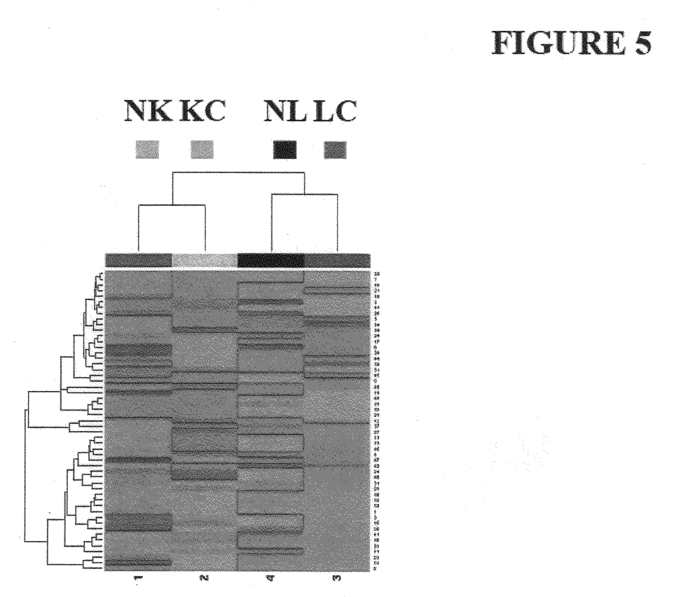

Significant degree of shared gene expression exists between tumors and their normal tissue counterparts. Expression analysis comparing different tumors may risk discover tissue differentiation markers indicative of lineage differentiation, which is irrelevant to the oncogenic process. This prompted us to investigate whether our 53 peptide biomarker panel indeed captured the “molecular portraits” of the assayed cancers rather than those of their paired normal tissue. We have performed two comparisons of either the kidney or liver tumor tissue and the adjacent normal tissue counterpart isolated from the same patient. In both kidney and liver cases, samples of the tumor or adjacent normal tissue counterpart from the same patient (FIG. 5) cluster together. However, in both the cases of the kidney and liver, expression profiles between the tumor and adjacent normal tissue counterpart were clearly very different. This analysis indicates that the 53 peptide biomarker panel not only discriminates multi-class cancer types but also delineates tumors from the adjacent normal tissue counterpart.

Our peptidomic biomarker discovery approach, which is commonly referred to as ion mapping, first selects biomarker candidate peaks on the basis of discriminant analysis and then targets them for MS/MS sequencing analysis. Peptides within the 53 peptide set have been subjected to extensive protein identification efforts via LTQ FT MS/MS and database searches upon both the tryptic and non-tryptic peptide fingerprinting analyses. Of the 53 peptide features (Table 1), 18 were positively identified where 5 peptides are non-tryptic and 13 are tryptic. Tryptic peptides of ALB, APOA1, and KRT8 were found in the 53 peptide biomarker panel, where the quantification analysis results of these peptides are in line with those obtained from previous spectrum counting analysis: three different tryptic ALB peptides were identified to have higher abundance in colon and liver cancer categories; one tryptic KRT8 peptides was identified to have higher abundance in colon cancer samples; one tryptic APO A1 were identified to have higher abundance in kidney cancer. Tryptic peptides from ADAM8, AGP2, IGKC, KRT18, MKI671P, and YWHAZ were also found. However, their parent proteins were shown to be undifferentiated by spectrum counting analysis. Non-tryptic peptides from ASB13, Cyclin-J, GP1BA, IGSF8, RUFY4, TRPM6, and ZSCAN4 were found to be part of the 53 peptide biomarker panel. Shown in Table 1, the 53 feature biomarker panel and each peptide biomarker's relative abundance the four assayed cancer types. CC, KC, LC, BC: colon, kidney, liver, and brain cancer s. m/z: mass to charge ratio. MH+: the molecular weight of the peptide with single positive charge. Retention time: HPLC fraction collection time.

| | | retention | | | | | | MH+ | | |
|---|---|---|---|---|---|---|---|---|---|---|
| index | m/z | time | CC | KC | LC | BC | charge | [Da] | sequence | gene |
| 52 | 647.26 | 223.63 | −0.08 | 0 | 0 | 0 | 3 | 1939.768 | FVERGEQCDCGPPEDCR | ADAM8 |
| 48 | 497.77 | 106.756 | 0 | 0.09 | 0 | 0 | 2 | 994.522 | TEDTIFLR | AGP2 |
| 18 | 717.79 | 119.133 | 0.345 | 0 | 0 | 0 | 2 | 1434.534 | ETYGEMADccAK | ALB |
| 23 | 675.83 | 92.018 | 0 | 0 | 0.28 | 0 | 2 | 1350.681 | SALEVDETYVPK | ALB |
| 36 | 588.26 | 145.455 | 0.169 | 0 | 0 | 0 | 3 | 1762.824 | AQYLQQcPFEDHVK | ALB |
| 26 | 554.26 | 73.8042 | 0 | 0 | 0 | 0.26 | 2 | 1107.514 | EAcMSVLLNA | ASB13 |
| 10 | 600.77 | 87.4973 | 0.393 | 0 | 0 | 0 | 3 | 1200.507 | HDGWPMICLE | Cyclin-J |
| 49 | 838.9 | 124.026 | 0 | 0 | 0 | 0.08 | 2 | 1676.866 | YTFSLATLMPYTRL | GP1BA |
| 29 | 625.97 | 8.4279 | 0.233 | 0 | 0 | 0 | 3 | 1875.935 | VYAcEVTHOGLSSPVTK | IGKC |
| 19 | 797.32 | 161.997 | 0 | 0 | 0 | 0.34 | 2 | 1593.636 | QDAGIYEcHTPSTD | IGSF8 |
| 43 | 521.31 | 92.8104 | 0.122 | 0 | 0 | 0 | 2 | 1041.607 | IVLQIDNAR | KRT18 |
| 41 | 626.81 | 104.612 | 0 | 0.12 | 0 | 0 | 2 | 1252.622 | VQPYLDDFQK | APO A1 |
| 6 | 672.84 | 123.216 | 0.492 | 0 | 0 | 0 | 2 | 1344.677 | ASLEAAIADAEQR | KRT8 |
| 14 | 773.93 | 178.672 | 0.383 | 0 | 0 | 0 | 2 | 1546.875 | IPFKQPSYPSVKR | MKI67IP |
| 51 | 642.31 | 223.736 | 0 | 0 | 0.08 | 0 | 2 | 1283.627 | RHPGLSLCSQW | RUFY4 |
| 8 | 670.36 | 127.317 | 0.405 | 0 | 0 | −0.1 | 2 | 1339.713 | IQNTFNFSLKQ | TRPM6 |
| 21 | 595.31 | 138.143 | 0.303 | 0 | 0 | 0 | 2 | 1189.662 | DSTLIMQLLR | YWHAZ |
| 7 | 761.9 | 232.505 | 0.416 | 0 | 0 | 0 | 3 | 1522.78 | KSSGKNLERFIED | ZSCAN4 |
| 1 | 586.32 | 195.579 | 0 | 0 | 0.98 | 0 | | | | |
| 2 | 1163.6 | 232.298 | 0.66 | 0 | 0 | 0 | | | | |
| 3 | 662.65 | 124.23 | 0 | 0 | 0.66 | 0 | | | | |
| 4 | 539.25 | 198.293 | 0 | 0 | 0.63 | 0 | | | | |
| 5 | 762.43 | 250.7 | 0.549 | 0 | 0 | 0 | | | | |
| 9 | 812.94 | 197.453 | 0.395 | 0 | 0 | 0 | | | | |
| 11 | 701.71 | 76.1418 | 0 | 0 | 0.39 | 0 | | | | |
| 12 | 625.87 | 111.571 | 0.389 | 0 | 0 | 0 | | | | |
| 13 | 522.23 | 0.941603 | 0.388 | 0 | 0 | 0 | | | | |
| 15 | 678.38 | 174.946 | 0 | 0 | 0.38 | 0 | | | | |
| 16 | 662.65 | 123.925 | 0 | 0 | 0.37 | 0 | | | | |
| 17 | 671.8 | 153.102 | 0.356 | 0 | 0 | 0 | | | | |
| 20 | 998.65 | 219.065 | 0 | 0 | 0.33 | 0 | | | | |
| 22 | 904.19 | 100.295 | 0.293 | 0 | 0 | 0 | | | | |
| 24 | 680.65 | 95.5725 | 0 | 0 | 0 | −0.3 | | | | |
| 25 | 891.23 | 241.136 | 0.267 | 0 | 0 | 0 | | | | |
| 27 | 878.75 | 89.0418 | 0 | 0 | 0.26 | 0 | | | | |
| 28 | 521.56 | 41.6975 | 0 | −0.1 | 0.25 | 0 | | | | |
| 30 | 762.4 | 24.7586 | 0.217 | 0 | 0 | 0 | | | | |
| 31 | 445.24 | 61.7389 | 0 | 0 | 0.2 | 0 | | | | |
| 32 | 476.78 | 239.368 | 0.196 | 0 | 0 | 0 | | | | |
| 33 | 696.98 | 158.166 | 0.187 | 0 | 0 | 0 | | | | |
| 34 | 404.2 | 65.121 | 0.178 | 0 | 0 | 0 | | | | |
| 35 | 819.48 | 1 | 0 | 0 | 0 | 0.17 | | | | |
| 37 | 780.42 | 125.569 | 0.156 | 0 | 0 | 0 | | | | |
| 38 | 758.81 | 44.3717 | 0 | 0 | 0.15 | 0 | | | | |
| 39 | 681.34 | 108.482 | 0 | 0 | 0 | −0.1 | | | | |
| 40 | 532.26 | 205.143 | 0.124 | 0 | 0 | 0 | | | | |
| 42 | 559.1 | 12.2807 | 0 | 0 | 0.12 | 0 | | | | |
| 44 | 601.26 | 14.4659 | 0.11 | 0 | 0 | 0 | | | | |
| 45 | 662.99 | 11.8647 | 0 | 0 | 0.1 | 0 | | | | |
| 46 | 797.82 | 14.7786 | 0 | −0.1 | 0 | 0.1 | | | | |
| 47 | 516.58 | 18.8344 | 0 | 0 | 0.1 | 0 | | | | |
| 50 | 1264.2 | 202.239 | 0.084 | 0 | 0 | 0 | | | | |
| 53 | 675.62 | 95.0574 | 0.066 | 0 | 0 | −0.1 | | | | |

### Discussion

Tumor associated proteins and peptides (TAP) are derived from tumor apoptosis/necrosis, secretion by tumor cells and tumor specific degradation of extracellular matrix proteins by proteases released from cancer cells. Besides being formed in the tumor, TAPs are released in the circulation, and therefore are resources as serological markers for early diagnosis, cancer relapse and metastasis, as well as for prognosis, site of tumor, and monitoring of therapy. Proteins and peptides from primary tumor cell conditioned media were subjected to extensive proteomic and peptidomic comparative analyses to discover tumor delineating biomarkers. The spectrum counting analysis led to the identification of a 12 protein biomarker panel including ALB, TF, APO A1, VIM, IGHG1, GFAP, A1AT, HBB, AGP1, PKM2, KRT8 and KRT19. We conclude that these 12 proteins have diagnostic significance in various tumors and are useful as serological biomarkers.

The cancer microenvironment can generate and shed tumor specific peptides, through the secretion of tumor specific proteases or protease inhibitors, which control the degradation of the surrounding proteins and/or extracellular matrix. The present invention provides a comprehensive peptidomic analysis, which identified a panel of 53 peptide biomarkers, including both tryptic and non-tryptic peptides, capable of discriminating between classes of tumors. The methods used herein are not influenced by low numbers of plasma high-abundance proteins; endogenous endoproteolytic and exoproteolytic enzymatic activity in serum; and artifacts of sample collection. Therefore, the tumor associated peptidomic patterns of the invention represent genuine differences between various tumors and their normal tissue counterparts.

In conclusion, the proteomic spectrum counting and peptidomic profilings of the invention have yielded two biomarker panels of 12 proteins and 53 peptides respectively, both capable of discriminating multiclass tumors. These tumor associated biomarkers can be assessed by antibody based or a quantitative mass spectrometry based approach for practical clinical utilities in serological diagnosis and prognosis.

### Methods

Samples: Tissue specimens used in this study were obtained with the approval of the Committee on the Ethical of Research involving Human Subjects from the Beijing 309 Hospital. The total 20-case sample group contained tumor samples from 3 colon cancer, 6 kidney cancer, 3 liver cancer, 4 glioblastma, 2 ureteral cancer patients, and normal organ samples from one kidney and one liver cancer patients. All samples were histologically confirmed by two independent pathologists. Following surgical resection, tumor tissues were cut into small pieces with sterile scissors and rinsed with PBS several times and placed in 50 ml conical tubes containing defined medium [Dulbecco's Modified Eagle Medium (DMEM/F12) supplemented with growth factor cocktail, which includes basic FGF 20 ng/ml, EGF 20 ng/ml, insulin 7 μg/ml and transferin 15 μg/ml, plus penicillin 500 units/ml and streptomycin 500 μg/ml]. Samples were cut into small pieces with sterile scissors and then incubated kept in the above defined medium overnight at 4° C. Following centrifugation for 10 minutes at 2000 rpm, the tissue media were desalted through the PD-10 column (GE health care) pre-equilibrated with 0.01% NH₄OH, then lyophilized and stored at −80° C.

Preparation of Tumor Associated Proteins: the Frozen Pellets were Sonicated and dissolved in 7 M urea and 2 M thiourea and 25 mM ammonium bicarbonate for 2 hours. The resulting protein extracts were desalted using Pierce zeba desalt spin columns. Each sample's total protein content was quantified by Pierce BCA protein assay reagent. The desalted samples were diluted with 25 mM ammonium bicarbonate to the same protein concentration 0.5 μg/μl. For reduction, 50 μg protein of each sample was incubated with 10 μl 5 mM DTT at 50° C. for 30 minutes. For alkylation, iodoacetamide was added to a final concentration of 15 mM. After incubating at room temperature in the dark for 30 minutes, 1 μg trypsin was added to each sample to digest at 37° C. overnight. 1.5 μl 50% TFA in water was added to terminate the reaction. The total volumes of the digests were subsequently dried to ˜70 μl in a SpeedVac.

LCMS and MSMS analysis: Trypsin digested and naturally occurring peptides were diluted, 1:10 in 0.1% v/v formic acid and loaded online to an analytical C18 column (75 μm, 12 cm). Peptides were eluted using a linear gradient of H₂O:CH₃CN (95:5, 0.1% formic acid buffer A) to H₂O:CH₃CN (70:30, 0.1% formic acid buffer B) at 300 nl/min over 70 minutes using a 2D Eskigent nano HPLC, Spark autosampler system. Each full MS scan (from 400 to 1600 m/z) acquired on an LTQ FTMS (Thermo, San Jose, Calif.) was followed by five MS/MS events using data-dependent acquisition where the first most intense ion from a given MS scan was subjected to CID followed by the second to fifth most intense ions. Protein identification was performed by searching SwissProt protein database using Thermal BioWorks™ software and SEQUEST® algorithm (Thermo, San Jose, Calif.). Peptide identifications were considered acceptable if they passed the thresholds determined acceptable for human plasma by Qian et al. and passed an additional filter of a PeptideProphet score of at least 0.7. The PeptideProphet score is representative of the quality of the SEQUEST™ identification and is based on a combination of XCorr, delCn, Sp, and a parameter that measures the probability that the identification occurred by random chance. PeptideProphet scores are normalized to a 0 to 1 scale, with 1 being the highest confidence value.

Spectrum counting analysis: Quantification of proteins in different samples was done by means of spectrum counting. From the MS/MS protein identifications, a separate list of proteins, was created for each sample, and the lists were then compared to find differential expressed proteins. Using Scaffold software (Proteome Software, Portland, Oreg.), spectrum counts were analyzed from the number of MS/MS spectra identified corresponding to each protein normalized to account for protein length or expected number of tryptic peptides. For any given protein, the relative abundance between samples was estimated by the comparative analysis of the normalized spectrum counts.

Peptidomic data analysis. Our approach, which is commonly referred to as ion mapping, first selects biomarker candidate MS peaks on the basis of discriminant analysis and then targets them for MS/MS sequencing analysis to obtain protein identification. Our in-house informatics platform, “MASSConductor”, an integrated suite of algorithms, statistical methods, and computer applications, has been specifically developed to allow adequate signal processing and statistical analysis in LCMS based urine peptide profiling. The software architecture of MASS-Conductor supports a farmed parallel process. A 30 node Linux cluster has been setup to support MASS-Conductor computational needs at the Stanford Medical School Data Center. The MS data can be viewed as a set of multidimensional discrete data points: the retention time dimension, the m/z dimension and the intensity dimension. A key step in data processing is to transform this large amount of raw data into a list of non-redundant cross category m/z features, while simultaneously tracking associated sample source, retention time, and intensity. When applied to each HPLC fraction MS spectrum from different sample categories, “MASS-Conductor” software extracts peaks from raw spectra, enables common peak alignment, generates “consensus” representative peaks across all spectra via two dimensional hierarchical clustering of both mass/charge and the HPLC fractions, and normalizes peak signal measurements. The peak finding algorithm was developed from previous work. We begin with the raw spectrum data. The raw spectra are smoothed using a “supersmoother”, which is helpful for locating peaks. The peak finding algorithm looks for sites (m/z values) whose intensity is higher than at the plus/minus 100-200 sites surrounding it and higher than the estimated average background at that site. The peak widths are ˜0.5% of the corresponding m/z value. Common peaks are defined by aligning peaks with m/z differences that are <0.05 and LC fraction number differences <30 simultaneously. The centroid of each cluster is extracted to represent the “consensus” position for that peak across all spectra. Empirically, it is most likely that two peptides with very different HPLC retention time but same m/z are different peptides. The binned LCMS peak data obtained for all samples of different categories.

What is claimed is:

1. A method for multiclass cancer discrimination of a patient sample, the method comprising:
determining a cancer biomarker distribution pattern from a patient sample of blood or a sample derived from blood, wherein said cancer biomarker distribution pattern comprises quantitative data for at least 10 proteins or at least 50 peptides;
comparing said cancer biomarker distribution pattern with control cancer biomarker distribution patterns indicative of cancer classes of interest for discrimination;
wherein a statistically significant match with a cancer biomarker distribution pattern for a cancer class of interest is indicative that said patient has a cancer of said class;
directing therapeutic intervention for said patient based on said multiclass cancer discrimination.

2. The method according to claim 1, wherein said cancer biomarker distribution pattern comprises quantitative data for the presence of at least 12 protein cancer biomarkers.

3. The method according to claim 2, wherein said protein cancer biomarkers include albumin (ALB), serotransferrin (TF), aplipoprotein A1 (APO A1), Vimentin (VIM), immunoglobulin heavy constant gamma 1(IGHG1), glial fibrillary acidic protein (GFAP), alpha 1 antitrypsin (A1AT), hemoglobin beta (HBB), orosomucoid 1 (AGP1, alias ORM1), pyruvate kinase type M2 (PKM2, alias M2-PK), keratin 8 (KRT8), and keratin 19 (KRT19).

4. The method of claim 3, wherein said quantitative data is obtained by a determination of specific antibody binding to said protein biomarkers.

5. The method of claim 3, wherein said quantitative data is obtained by a quantitative mass spectrometry.

6. The method according to claim 1, wherein said cancer biomarker distribution pattern comprises quantitative data for the presence of at least 53 peptide cancer biomarkers.

7. The method according to claim 6 wherein said peptides include peptides identified in Table 1.

8. The method according to claim 7, wherein said quantitative data is obtained by a determination of specific antibody binding to said peptide biomarkers.

9. The method of claim 7, wherein said quantitative data is obtained by a quantitative mass spectrometry.

10. The method of claim 1, wherein said cancer classes of interest include brain cancer, kidney cancer, liver cancer and colon cancer.

11. An array comprising binding agents specific for at least 10 cancer biomarkers.

12. The array of claim 11, wherein said cancer biomarkers include albumin (ALB), serotransferrin (TF), aplipoprotein A1 (APO A1), Vimentin (VIM), immunoglobulin heavy constant gamma 1 (IGHG1), glial fibrillary acidic protein (GFAP), alpha 1 antitrypsin (A1AT), hemoglobin beta (HBB), orosomucoid 1 (AGP1, alias ORM1), pyruvate kinase type M2 (PKM2, alias M2-PK), keratin 8 (KRT8), and keratin 19 (KRT19).

13. The array of claim 11, wherein said cancer biomarkers include peptide cancer biomarkers set forth in Table 1.

14. A kit for use in the methods of claim 1, comprising reagents that specifically identify cancer biomarkers of the invention; and instructions for use.
